(19) United States
(12) Patent Application Publication
(10) Pub. No.: US 2016/0067169 A1
(43) Pub. Date: Mar. 10, 2016

(54) TOOTHPASTE CONTAINING FLUORIDE AND POLYLACTIC ACID PARTICLES

(71) Applicant: Henkel AG & Co. KGaA, Duesseldorf (DE)

(72) Inventors: Thomas Welss, Duesseldorf (DE); Thomas Foerster, Duesseldorf (DE); Markus Semrau, Langenfeld (DE); Kristin Miehlich, Wuppertal (DE); Claudia Hundeiker, Meerbusch (DE)

(21) Appl. No.: 14/944,875

(22) Filed: Nov. 18, 2015

Related U.S. Application Data
(63) Continuation of application No. PCT/EP2014/052032, filed on Feb. 3, 2014.

(30) Foreign Application Priority Data
May 28, 2013 (DE) 10 2013 209 899.5

(51) Int. Cl. A61K 8/85; A61Q 11/00; A61K 8/21

(52) U.S. Cl. CPC A61K 8/85; A61K 8/21; A61Q 11/00; A61K 2800/49; A61K 2800/92; A61K 2800/413; A61K 2800/412; A61K 2800/28; A61K 2800/596

(57) ABSTRACT

Oral and dental hygiene and cleaning agents, which contain, relative to the weight thereof, 0.001 to 25 wt % polylactic acid particles and 1200 to 1600 ppm fluoride have a substantially improved degree of fluoride deposition and offer an optimum compromise between applicability and plaque reduction.

### FIELD OF THE INVENTION

The present invention generally relates to a dentifrice, i.e., toothpaste that promotes plaque reduction and fluoride deposition and also takes into account the special requirements in the use of electric toothbrushes.

### BACKGROUND OF THE INVENTION

Dental cleaning agents are on the market in a variety of forms and serve primarily to clean the dental surface and to prevent diseases of the teeth and gingiva. They usually contain a combination of polishing agents, humectants, surfactants, binders, flavorings and active ingredients that contain fluoride and antimicrobial active ingredients. In addition to tooth powders, which play a lesser role because of their high abrasiveness, dental cleaning agents are available primarily in the form of pastes, creams and translucent or transparent gels. Liquid toothpastes and mouthwashes have become increasingly important in recent years.

In parallel with this, further developments have also been pursued with regard to the equipment. In addition to the traditional manual toothbrush, which is moved by the user with circular motions in the mouth, electric toothbrushes have also become established on the market, creating some of the movement of the bristles on the tooth surface by means of a battery—usually rechargeable—located in the hand piece. Therefore, depending on the model, the user can replace the circular hand motion with a horizontal linear motion.

Electrically driven brush heads are customary on the market in a variety of embodiments. Thus there are round brush heads which are induced to rotate clockwise and counterclockwise in rotation or partial rotation. However, there are also elongated brush heads modeled on the basis of the traditional toothbrush that oscillate electrically.

All electric toothbrushes have the advantage that they relieve the user of a portion of certain movement sequences and have the reputation of cleaning the teeth more thoroughly. However these advantages should also be considered in comparison with disadvantage: an electric toothbrush usually results in a more intense load on the tooth surface than that in tooth brushing by hand. If the type and shape of the abrasive substances in the toothpaste are not adjusted for this purpose, there may be damage to the dentin in the use of an electric toothbrush.

Another problem is that the complex mechanism in the brush head can become stuck and the toothbrush can then no longer execute the corresponding electric movement. In addition, the rheology can also be taken into account because there can be a certain adhesive power of the brush heads in movement to avoid spraying the toothpaste.

Some of the requirements listed above are in contradiction with one another because abrasive substances are at least proportionally also consistency-producing agents.

In the use of traditional toothbrushes, the rheology of the tooth cleaning agent also plays an important role. The viscosity of the composition should be high enough to allow it to be applied to the tooth brush but during brushing it should promptly result in an application mixture in the oral cavity that permits a high fluoride deposition, which can usually be accomplished more effectively with low viscosity compositions.

Another field of goals consists of providing products which achieve the goals assigned to them while having the lowest possible consumption of raw materials in order to save on resources and be able to provide ecologically acceptable products. Reducing synthetic ingredients is therefore another important complex of goals in the formulation of cosmetic agents.

The use of polylactic acid particles in products for oral hygiene is described in the International Patent Applications WO2012/177616 and WO2012/177617, but the particles are disclosed there exclusively as biodegradable abrasive substances. These patent applications do not contain any information about fluoride deposition, and furthermore, fluoride is used only in amounts of <1100 ppm in the exemplary compositions.

The object of the present invention was therefore to provide an oral and dental hygiene and cleaning agent that would solve the range of problems defined above and would have a sufficient viscosity to be applicable to brush heads of traditional or electric toothbrushes but still have a low enough viscosity to permit effective plaque reduction and a high fluoride deposition. Furthermore, the formulations should contain the fewest possible synthetic ingredients.

It has been found that the use of polylactic acid particles in compositions with a fluoride content above 1200 ppm leads to a definitely improved fluoride deposition, and furthermore, an optimum compromise between applicability and plaque reduction is achieved. In addition, these compositions are especially suitable for cleaning by means of electric toothbrushes because they protect the sensitive mechanism of electrically operated brush heads.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Oral and dental hygiene and cleaning agent including—based on its weight—0.001 to 25 wt % polylactic acid particles, and 1200 to 1600 ppm fluoride.

Use of polylactic acid particles to increase the fluoride deposition.

### DETAILED DESCRIPTION OF THE INVENTION

The following detailed description of the invention is merely exemplary in nature and is not intended to limit the invention or the application and uses of the invention.

Furthermore, there is no intention to be bound by any theory presented in the preceding background of the invention or the following detailed description of the invention.

In a first embodiment, the invention relates to an oral and dental hygiene and cleaning agent that includes—based on its weight:

a) 0.001 to 25% by weight polylactic acid particles, and
b) 1200 to 1600 ppm fluoride.

Oral and dental care agents as well as oral and dental cleaning agents in the sense of the invention include oral and dental powders, oral and dental pastes, liquid oral and dental creams, oral and dental rinses as well as oral and dental gels. Toothpastes and liquid dental cleaning agents are especially suitable here. The oral and dental hygiene and cleaning agents may be in the form of toothpastes, liquid toothpastes, dental powders, mouthwashes or optionally even a chewable composition, for example, chewing gum. However, they are preferably present in the form of more or less flowable or plastic toothpastes, such as those used for cleaning teeth with a toothbrush. Another preferred embodiment of the present invention is for mouthwash and mouth rinse solutions that can be used for rinsing out the oral cavity.

The first important ingredient contained in the compositions according to the invention, based on weight, is 0.001 to 25% by weight polylactic acid particles.

Preferred agents according to the invention use polylactic acid particles within narrower quantity ranges. Oral and dental hygiene and cleaning agents according to the invention here preferably contain—based on their weight—0.002-20% by weight, preferably 0.003-17.5% by weight, especially preferably 0.004 to 15% by weight, extremely preferably 0.005-12.5% by weight and in particular 0.01 to 10% by weight polylactic acid particles.

Polylactic acid, also known as polylactide or PLA, is a term referring to biodegradable polymers (polyesters), which can be obtained primarily by ionic polymerization of lactide, which involves ring closure of two lactic acid molecules.

A ring-opening polymerization takes place at temperatures between 140 and 180° C. as well as under the influence of catalytic tin compounds (e.g., tin oxide). This produces plastics with a high molecular weight and strength. Lactide itself can be produced by fermentation of molasses or fermentation of glucose with the help of various bacteria.

In addition, high-molecular and pure polylactides can be produced directly from lactic acid by means of so-called polycondensation. However, disposal of the solvent is problematical in industrial production.

Lactic acid (2-hydroxypropanoic acid) has an asymmetrical carbon atom, so that the polylactic acid also has optically active centers in L(+) and D(−) configurations. The ratio of L monomer units to D monomer units determines the degree of crystallinity, the melting point and the biodegradability of the polymers.

Suitable polylactic acids according to the invention include L-polylactic acid, D-polylactic acid and L/D-polylactic acid as well as mixtures thereof. L-Polylactic acid is especially preferred because of its very good biodegradability. In a preferred embodiment of the present invention, the amount by weight of L-lactic acid monomer units in the polylactic acid is greater than 50% by weight, preferably greater than 80% by weight and in particular greater than 90% by weight.

The molecular weight of the polylactic acid is usually 1000 to 1,000,000, preferably 10,000 to 300,000, more preferably 50,000 to 250,000 and in particular from 100,000 to 180,000 Dalton.

In another preferred embodiment of the present invention, the polylactic acid is used in a form that has been blended with fillers. The use of larger amounts of fillers helps to reduce the polymer to particles, increases the biodegradability as well as the internal specific surface area by way of the porosity and capillarity. Water-soluble fillers are preferred here in particular, for example, metal chlorides such as NaCl, KCl, etc., meta 1 carbonates such as Na₂CO₃, NaHCO₃, etc., metal sulfates such as MgSO₄.

The fillers used may also be natural raw materials, for example, nut shells, wood or bamboo fibers, starch, xanthan, alginates, dextran, agar, etc. These fillers are biodegradable and do not have a negative effect on the good ecological properties of polylactic acid particles.

The biodegradable filler content of the polylactic acid particles may usually be 10 to 70% by weight, wherein amounts of 20 to 60% by weight are preferred and amounts of 30 to 50% by weight are especially preferred.

Even if the polylactic acid particles are not used because of their abrasive properties within the scope of the present invention, irregular shapes have proven to be especially preferred because the effect according to the invention can be increased further in comparison with spherical particles.

Especially suitable particles according to the invention have a circularity between 0.1 and 0.6.

The shape of the polylactic acid particles used according to the invention can be defined in various ways, but within the scope of this preferred embodiment of the present invention, the geometric proportions of a particle and—pragmatically—a particle aggregate can be determined.

Recent high-precision methods allow accurate determination of particle shapes from a large number of particles, usually of more than 10,000 particles, preferably more than 100,000 particles. These methods permit an accurate selection of the average particle shape of a particle aggregate. The particle shapes are preferably determined using an “Occhio Nano 500 Particle Characterization Instrument” with the “Callistro version 25” software (Occhio s.a., Liège, Belgium). This device permits preparation, dispersion, imaging and analysis of a particle aggregate with the device parameters preferably being set as follows: white requested=180, vacuum time=5000 ms, sedimentation time=5000 ms, automatic threshold, number of particles counted/analyses=8000 to 500,000, minimum number of replicates/sample=3, lens setting 1×/1.5×.

The polylactic acid particles that can be used according to the invention preferably have sizes characterized by their equivalent area diameter (ISO 9276-6:2008 (E) section 7) also known as the “equivalent circle diameter (ECD)” (ASTM F1877-05, section 11.3.2). The average ECD of a particle aggregate is calculated as the average ECD of each individual particle of a particle aggregate of at least 10,000 particles, preferably more than 50,000 particles, in particular more than 100,000 particles, after excluding particles with an equivalent area diameter (ECD) of less than 10 μm from the measurement.

In a preferred embodiment of the present invention, the polylactic acid particles have average ECD values of 10 to 100,000 μm and/or of 50 to 500 μm, more preferably of 100 to 350 μm and in particular of 150 to 250 μm.

Regardless of the average particle size, oral and dental hygiene and cleaning agents according to the invention, in which the polylactic acid particles have absolute particle sizes of 1 to 1000 μm, preferably of 2 to 750 μm and in particular of 10 to 500 μm are preferred.

Within the scope of the present invention, shape descriptors, which are calculations from geometric descriptors and/or form factors, are used. Form factors are ratios between two different geometric properties, which in turn comprise a measurement of the proportions of the image of an entire particle or the measurement of the proportions of an ideal geometric body enclosing the particles.

These results are descriptors, which are similar to size ratios (aspect ratios).

In a preferred embodiment of the present invention, meso-form descriptors are used to characterize particles. These meso-form descriptors indicate the extent to which a particle deviates from an ideal geometric shape, in particular from a sphere. In the preferred embodiment of the present invention, the polylactic acid particles are different from the typical spherical shape or spheroidal shapes such as granular particles, for example.

The particles here preferably have sharp edges and corners and preferably have concave indentations. Sharp edges of nonspherical particles may be defined as having a radius of less than 20 μm, preferably less than 8 μm and in particular less than 5 μm, wherein the radius is defined as the radius of an imaginary circle following the contour of the corner.

Circularity is a quantitative two-dimensional image analysis and can be determined according to ISO 9276-6:2008(E) section 8.2. Circularity is a preferred meso-form descriptor and can be determined, for example, using the “Occhio Nano 500 Particle Characterization Instrument” described above with the “Callistro version 25” software (Occhio s.a., Liège, Belgium) or the “Malvern Morphologi G3.” Circularity is occasionally described in the literature as the difference between a particle and a perfect spherical shape. The values for circularity vary between 0 and 1, where 1 describes a perfect sphere or (in a two-dimensional image) a perfect circle:

C=[(4πA)/P²]^{1/2}

where A denotes the projected area (the two-dimensional descriptor), and P denotes the length of the perimeter of the particle.

Polylactic acid particles with an average circularity C of 0.1 to 0.6, preferably of 0.15 to 0.4, and in particular of 0.2 to 0.35 have proven to be particularly suitable within the scope of the present invention. The average values here are obtained by forming a quotient of volume-based measurements and number-based measurements.

Solidity is a quantitative two-dimensional image analysis and can be determined according to ISO 9276-6:2008(E) section 8.2. Solidity is also a preferred meso-form descriptor and can be determined using the “Occhio Nano 500 Particle Characterization Instrument” described above with the “Callistro version 25” software (Occhio s.a., Liège, Belgium) or the “Malvern Morphologi G3.” Solidity is a meso-form descriptor, which describes the concavity of a particle and/or of a particle aggregate. The values for solidity vary between 0 and 1, where 1 describes a non-concave particle:

Solidity=A/Ac,

where A describes the (image) area of the particle, and Ac describes the area of the convex shell enclosing the particle.

Polylactic acid particles with an average solidity of 0.4 to 0.9, preferably of 0.5 to 0.8, and in particular 0.55 to 0.65 have proven to be particularly suitable within the scope of the present invention. The average values here are obtained by forming a quotient of volume-based measurements and number-based measurements.

In especially preferred embodiments of the present invention, the polylactic acid particles used have an average circularity C of 0.1 to 0.6, preferably of 0.15 to 0.4, and in particular of 0.2 to 0.35, and an average solidity of 0.4 to 0.9, preferably of 0.5 to 0.8, and in particular of 0.55 to 0.65.

The “average” circularities and solidities are average values from measurements on a large number of particles, usually more than 10,000 particles, preferably more than 50,000 particles and in particular more than 100,000 particles, where particles with an equivalent circle diameter (ECD) of less than 10 μm are excluded from the measurement.

After synthesis, the polylactic acid polymer can be converted to the desired particle size and shape by milling methods, for example.

An especially preferred method of producing particles of the desired circularity and solidity is by producing a foam of polylactic acid and then milling it.

Even if the polylactic acid particles are not used within the scope of the present invention because of their abrasive properties, polylactic acid particles of a certain hardness have proven to be particularly preferred because the effect according to the invention can be further enhanced with these particles in comparison with particles that are too hard or too soft.

The hardness of the particles can be varied by the ratio of D monomers to L monomers and by the molecular weight.

Preferred polylactic acid particles have a hardness of 3 to 50 kg/mm², preferably of 4 to 25 kg/mm² and in particular of 5 to 15 kg/mm² on the HV Vickers hardness scale.

The agents according to the invention contain fluoride as the second essential ingredient. This can be supplied in the form of inorganic fluoride salts (sodium fluoride, stannous fluoride, sodium monofluorophosphate, etc.); amine fluorides such as Olaflur are also suitable.

It has surprisingly been found that fluoride deposition can be increased if polylactic acid particles are also present in the compositions above certain fluoride contents. The minimum amount of fluoride here is 1200 ppm. Below this limit, the use of polylactic acid does not have a noticeable effect on fluoride deposition.

Oral and dental hygiene and cleaning agents preferred according to the invention are characterized in that they contain 1225 to 1575 ppm, preferably 1250 to 1550 ppm, more preferably 1275 to 1525 ppm, even more preferably 1300 to 1500 ppm, even more preferably 1325 to 1475 ppm and in particular 1350 to 1450 ppm fluoride.

If fluoride is supplied in the form of sodium fluoride, 1% by weight sodium fluoride corresponds to approximately 4524 ppm fluoride, so that preferred agents according to the invention contain 0.27 to 0.35% by weight, preferably 0.28 to 0.34% by weight, more preferably 0.29 to 0.33% by weight and in particular 0.30 to 0.32% by weight sodium fluoride.

The oral and dental hygiene and cleaning agents according to the invention contain additional ingredients. The use of so-called humectants, which prevent drying of toothpastes, is preferred here. With so-called liquid toothpastes having a flowable rheology, these serve as the matrix and are used in larger amounts. In the case of mouthwashes and mouth rinses, these alcohols serve as consistency regulators and also as sweeteners.

Oral and dental hygiene and cleaning agents according to the invention which contain—based on their weight—0.5 to 60% by weight, preferably 0.75 to 55% by weight, especially preferably 1 to 50% by weight and in particular 2 to 40% by weight of at least one polyvalent alcohol from the group of sorbitol and/or glycerol and/or 1,2-propylene glycol or mixtures thereof are preferred according to the invention.

For certain application ranges, it may be advantageous to use just one of the three ingredients listed above. Sorbitol is preferred in most cases, but mixtures of two of the three substances or all three substances may be preferred in other application fields. A mixture of glycerol, sorbitol and 1,2-propylene glycol in a weight ratio of 1:(0.5-1):(0.1-0.5) has proven to be particularly advantageous here.

In addition to sorbitol and/or glycerol and/or 1,2-propylene glycol, additional polyvalent alcohols that are suitable here include those having at least two OH groups, preferably mannitol, xylitol, polyethylene glycol, polypropylene glycol and mixtures thereof. Of these compounds, those with two to 12 OH groups and in particular those with 2, 3, 4, 5, 6 or 10 OH groups are preferred.

Polyhydroxy compounds with two OH groups include, for example, glycol (CH₂(OH)CH₂OH) and other 1,2-diols such as H—(CH₂)ₙ—CH(OH)CH₂OH where n=2, 3, 4, 5, 6, 7, 8, 9, 10, 11, 12, 13, 14, 15, 16, 17, 18, 19, 20. 1,3-Diols such as H—(CH₂)ₙ—CH(OH)CH₂CH₂OH, where n=1, 2, 3, 4, 5, 6, 7, 8, 9, 10, 11, 12, 13, 14, 15, 16, 17, 18, 19, 20 can also be used according to the invention. The (n, n+1)-diols and/or (n, n+2)-diols with non-terminal OH groups may also be used. Important representatives of the polyhydroxy compounds having two OH groups also include the polyethylene and polypropylene glycols. Additional polyvalent alcohols that may be use include, for example, xylitol, propylene glycols, polyethylene glycols, in particular those with an average molecular weight of 200-800.

The use of sorbitol is especially preferred, so agents including no polyvalent alcohols other than sorbitol are especially preferred.

The oral and dental hygiene agents, in particular the toothpastes, may also contain substances to make the teeth less sensitive, for example, potassium salts, e.g., potassium nitrate, potassium citrate, potassium chloride, potassium bicarbonate and potassium oxalate. Oral and dental hygiene and cleaning agents that are preferred according to the invention are characterized in that they contain substances to make teeth less sensitive, preferably potassium salts, especially preferably potassium nitrate and/or potassium citrate and/or potassium chloride and/or potassium bicarbonate and/or potassium oxalate are present, preferably in amounts of 0.5 to 20% by weight, especially preferably from 1.0 to 15% by weight, more preferably from 1.5 to 5% by weight and in particular from 1.75 to 2.5% by weight, each based on the total agent.

The agents according to the invention may also contain other wound healing and anti-inflammatory substances, e.g., active ingredients against gingivitis. Such substances may be selected, for example, from allantoin, azulene, chamomile extract, tocopherol, panthenol, bisabolol, sage extracts.

The use of abrasive substances is also preferred. Abrasives are amorphous, primarily inorganic, largely water-insoluble, extremely small-particle powders without sharp edges. They promote cleaning of the teeth in oral and dental hygiene agents while polishing the tooth surface at the same time (polishing agent).

Suitable polishing agents include in principle all friction bodies known for toothpaste, in particular those that do not contain any calcium ions. Preferred suitable polishing agent components therefore include silicic acids, aluminum hydroxide, aluminum oxide, sodium aluminum silicates, organic polymers or mixtures of such abrasive substances.

Polishing components that contain calcium, such as chalk, calcium pyrophosphate or dicalcium phosphate dihydrate, however, may be present in amounts of up to 5% by weight, based on the total composition.

The total amount of the polishing agents is preferably in the range of 5-50% by weight of the dental hygiene agent.

Toothpastes and liquid tooth cleaning agents including silicic acids as the polishing agent are especially preferred. Suitable silicic acids include, for example, gel silicic acids, hydrogel silicic acids and precipitated silicic acids. Gel silicic acids are produced by reacting sodium silicates solutions with strong aqueous mineral acids, forming a hydrosol, aging to form the hydrogel, then washing and drying. If the gel is dried to a water content of less than 15% by weight, irreversible shrinkage of the previously loose structure of the hydrogel occurs to from the dense structure of the so-called xerogel.

Precipitated silicic acids are a second group of suitable and preferred silicic acid polishing agents obtained by precipitation of silicic acid from dilute alkali silicate solutions by adding strong acids under conditions, under which aggregation to form the sol and the gel cannot occur. Suitable methods for preferably suitable is a precipitated silicic acid with a BET surface area of 15-110 m²/g, a particle size of 0.5-20 μm, wherein at least 80% by weight of the primary particles should be less than 5 μm, and the viscosity in 30% glycerol:water (1:1) dispersion of 30-60 Pa·s (20° C.) in an amount of 10-20% by weight of the toothpaste. Preferred suitable precipitated silicic acids of this type also have rounded corners and edges and are available under the brand name Sident® 12 DS (Degussa).

Other precipitated silicic acids of this type include Sident® 8 (Degussa) and Sorbosil® AC 39 (Crosfield Chemicals). These silicic acids are characterized by a lower thickening effect and a somewhat larger average particle size of 8-14 μm with a specific surface area of 40-75 m²/g (according to BET) and are particularly suitable for liquid toothpastes. These should have a viscosity of 10-100 Pa·s (25° C., shear rate D=10 s⁻¹).

However, toothpastes having a much higher viscosity of more than 100 Pa·s (25° C., D=10 s⁻¹) require a sufficiently large amount silicic acid with a particle size of less than 5 μm, preferably at least 3% by weight of a silicic acid with a particle size of 1-3 μm. Therefore, more finely divided, so-called thickening silicic acids with a BET surface area of 150-250 m²/g, for example, the commercial products Sipernat® 22 LS or Sipernat® 320 DS, are preferably also added to such toothpastes in addition to the precipitated silicic acids mentioned above.

For example, aluminum oxide in the form of weakly calcined alumina with a content of—and—aluminum oxide in an amount of approx. 1-5% by weight may also be present as an additional polishing agent component. Such a suitable aluminum oxide is available under the brand name “Poliertonerde P10 feinst” [ultrafine polishing alumina P10] (Giulini Chemie).

Additional suitable polishing agent components are available under the brand name “Pumice” (including Hess, Grolman).

In addition, all abrasive substances known for toothpastes are also suitable for use as the polishing agent such as sodium aluminum silicates, e.g., zeolite A, organic polymers, e.g., polymethacrylates or mixtures of these and the abrasive substances listed above.

In summary, preferred oral and dental hygiene and cleaning agents according to the invention also contain abrasives, preferably silicic acids, aluminum hydroxide, aluminum oxide, calcium pyrophosphate, chalk, dicalcium phosphate dihydrate (CaHPO₄.2H₂O), sodium aluminosilicates, in particular zeolite A, organic polymers, in particular polymethacrylates or mixtures of these abrasive substances, preferably in amounts of 1 to 30% by weight, preferably 2.5 to 25% by weight and in particular 5 to 22% by weight, each based on the total agent.

Especially preferred oral and dental hygiene and cleaning agents according to the invention contain—based on their weight—5 to 25% by weight, preferably 7.5 to 22.5% by weight, more preferably 10 to 20% by weight and in particular 12.5 to 18% by weight polishing agent from the group of silicic acids, aluminum hydroxide, aluminum oxide, dicalcium phosphate dihydrate (CaHPO₄.2H₂O) or mixtures of these abrasive substances.

The stated amounts are based on the total amount of abrasive substance, wherein individual abrasive substances are preferably used in narrower quantity ranges. Preferred agents according to the invention contain, for example, 5 to 20% by weight, preferably 8 to 21% by weight, more preferably 9 to 20% by weight and in particular 11 to 19% by weight silicic acid(s). Additional preferred agents according to the invention are characterized in that they contain 0.25 to 2% by weight, preferably 0.5 to 1.5% by weight and in particular 0.75 to 1.25% by weight aluminum oxide.

In summary, oral and dental hygiene and cleaning agents according to the invention which additionally contain polishing substances, preferably silicic acids, aluminum hydroxide, aluminum oxide, calcium pyrophosphate, chalk, dicalcium phosphate dihydrate (CaHPO₄.2H₂O), sodium aluminum silicates in particular zeolite A, organic polymers, in particular polymethacrylates or mixtures of these abrasive substances are preferred according to the invention, preferably in amounts of 1 to 30% by weight, preferably from 2.5 to 25% by weight and in particular from 5 to 22% by weight, each based on the total agent.

Oral and dental hygiene and cleaning agents may also contain other substances that are effective against plaque and/or dental calculus.

Substances that are effective against dental calculus may include, for example, chelating agents such as ethylenediaminetetraacetic acid and its sodium salts, phosphate salts such as the water-soluble dialkali or tetraalkali metal pyrophosphate salts, e.g., Na₄P₂O₇, K₄P₂O₇, Na₂K₂P₂O₇, Na₂H₂P₂O₇ and K₂H₂P₂O₇ or polyphosphate salts, which may be selected from water-soluble alkali metal tripolyphosphates such as sodium tripolyphosphate and potassium tripolyphosphate, for example.

Oral and dental hygiene and cleaning agents that are preferred according to the invention are characterized in that they additionally contain phosphate(s), preferably alkali metal phosphate(s) and in particular sodium tripolyphosphate, preferably in amounts of 1 to 10% by weight, especially preferably of 2 to 8% by weight and in particular of 3 to 7% by weight, each based on the total agent.

For example, natural and/or synthetic water-soluble polymers such as alginates, carraghenates, gum tragacanth, starch and starch ethers, cellulose ethers such as, for example, carboxymethyl cellulose (sodium salt), hydroxyethyl cellulose, methyl hydroxypropyl cellulose, guar gum, acacia gum, agar, xanthan gum, succinoglycan gum, carob bean powder, pectins, water-soluble carboxyvinyl polymers (e.g., Carbopol® products), polyvinyl alcohol, polyvinylpyrrolidone, polyethylene glycols, in particular those with molecular weights of 1500 to 1,000,000.

Additional substances that are suitable for controlling viscosity include, for example, sheet silicates such as montmorillonite clays, colloidal thickening silicic acids such as aerogel silicic acids, pyrogenic silicic acids or extremely finely milled precipitated silicic acids. Viscosity stabilizing additives from the group of cationic, zwitterionic or ampholytic nitrogen-containing surfactants, the hydroxypropyl-substituted hydrocolloids or polyethylene glycol/polypropylene glycol copolymers having an average molecular weight of 1000 to 5000 or a combination of the compounds mentioned above may also be used in the toothpastes.

Surface-active substances can also be used in the agents according to the invention. They serve to support the cleaning effect in toothpastes, for example, and if desired also serve to develop foam for toothbrushes or for mouthwashes as well as to stabilize the polishing body dispersion in the carrier and are usually used in mouthwash solutions as well as in toothpastes in an amount of 0.1-5% by weight.

Suitable surfactants include, for example, linear sodium alkyl sulfates with 12-18 carbon atoms in the alkyl group. These substances additionally have an enzyme-inhibiting effect on the bacterial metabolism of dental plaque. Other suitable surfactants include alkali salts preferably sodium salts of alkyl polyglycol ether sulfate with 12-16 carbon atoms in the linear alkyl group and 2-6 glycol ether groups in the molecule, of linear alkane (C₁₂-C₁₈) sulfonate, of sulfosuccinic acid monoalkyl (C₁₂-C₁₈) esters, of sulfated fatty acid monoglycerides, sulfated fatty acid alkanolamides, sulfoacetic acid alkyl (C₁₂-C₁₆) esters, acyl sarcosines, acyl taurides and acyl isothionates each with 8-18 carbon atoms in the acyl group. Zwitterionic, ampholytic and nonionic surfactants are also suitable, e.g., ethoxylates of fatty acid mono- and diglycerides, of fatty acid sorbitan esters and alkyl (oligo)glucosides, as well as fatty acid amidoalkylbetaines.

A preferred group of surfactants to be used are the anionic surfactants. Preferred oral and dental hygiene and cleaning agents according to the invention are characterized in that they contain 0.25 to 4% by weight, preferably 0.5 to 3.5% by weight, more preferably 0.75 to 3% by weight, even more preferably 1 to 2.5% by weight and in particular 1.6 to 2.2% by weight anionic surfactant(s).

Typical examples of anionic surfactants includes soaps, alkyl benzene sulfonates, alkane sulfonates, olefin sulfonates, alkyl ether sulfonates, glycerol ether sulfonates, α-methyl ester sulfonates, sulfofatty acids, alkyl sulfates, fatty alcohol ether sulfates, glycerol ether sulfates, hydroxy mixed ether sulfates, monoglyceride (ether) sulfates, fatty acid amide (ether) sulfate, mono- and dialkyl sulfosuccinates, mono- and dialkylsulfosuccinamates, sulfotriglycerides, amide soaps, ether carboxylic acids and salts thereof, fatty acid isethionates, fatty acid sarcosinates, fatty acid taurides, N-acylamino acids such as acyl lactylates, acyl tartrates, acyl glutamates and acyl aspartates, alkyl oligoglucoside sulfates, protein fatty acid condensates (in particular vegetable products based on wheat) and alkyl (ether) phosphates, for example. If the anionic surfactants contain polyglycol ether chains, they may have a conventional homolog distribution but they preferably have a narrow homolog distribution.

Most especially preferred agents according to the invention contain alkyl sulfate(s) as the anionic surfactant. Especially preferred oral and dental hygiene and cleaning agents according to the invention here are characterized in that they contain 0.25 to 4% by weight, preferably 0.5 to 3.5% by weight, more preferably 0.75 to 3% by weight, even more preferably 1 to 2.5% by weight and in particular 1.6 to 2.2% by weight sodium dodecyl sulfate.

The agents according to the invention especially preferably contain in addition or as an alternative to the anionic surfactants, amphoteric surfactant(s). Ampholytic surfactants and emulsifiers are understood to be those surface-active compounds which also contain at least one free amino group in addition to a C₈-C₂₄ alkyl or acyl group and have at least one COOH or SO₃H group and are capable of forming internal salts. Examples of suitable ampholytic surfactants include N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with approx. 8 to 24 carbon atoms in the alkyl group. Especially preferred ampholytic surfactants include N-coconut alkylaminopropionate, coconut acylaminoethylaminopropionate and C₁₂-C₁₈ acyl sarcosines.

Especially preferred oral and dental hygiene and cleaning agents are characterized in that they contain 0.01 to 2% by weight, preferably 0.05 to 1.5% by weight, more preferably 0.1 to 1% by weight, even more preferably 0.12 to 0.7% by weight and in particular 0.15 to 0.6% by weight amphoteric surfactant(s).

Especially preferred oral and dental hygiene and cleaning agents according to the invention are characterized in that they contain amphoteric surfactant(s) from the groups of
N-alkylglycines,
N-alkylpropionic acids,
N-alkylaminobutyric acids,
N-alkyliminodipropionic acids,
N-hydroxyethyl-N-alkylamidopropylglycines,
N-alkyltaurines,
N-alkylsarcosines,
2-alkylaminopropionic acids with, each with approx. 8 to 24 carbon atoms in the alkyl group,
alkylaminoacetic acids each with approx. 8 to 24 carbon atoms in the alkyl group,
N-cocoalkylaminopropionate,
Cocoacylaminoethylaminopropionate,
N-alkyl-N,N-dimethylammonium glycinates, for example, cocoalkyldimethylammonium glycinate,
N-acylaminopropyl-N,N-dimethylammoniumglycinates, for example, cocoacylaminopropyldimethylammonium glycinate,
2-alkyl-3-carboxymethyl-3-hydroxyethylimidazoline, each with 8 to 18 carbon atoms in the alkyl or acyl group,
cocoacylaminoethylhydroxyethylcarboxymethyl glycinate,
the compounds known under the INCI designation cocamidopropylbetaine
the compounds under the INCO designation disodium cocoamphodiacetate.

Especially preferred oral and dental hygiene and cleaning agents contain as the amphoteric surfactants betaines of the formula (Bet-I)

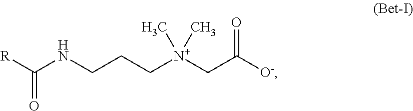

in which R stands for a linear or branched, saturated or mono- or polyunsaturated alkyl or alkenyl radical with 8 to 24 carbon atoms.

These surfactants are referred to according to the INCI nomenclature as amidopropylbetaines, where the representatives derived from coconut fatty acids are preferred and are known as cocoamidopropylbetaines. According to the invention surfactants of the formula (Bet-I) that are a mixture of the following representatives are especially preferred:
H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Surfactants of the formula (Bet-I) are especially preferably used within narrower quantity ranges. Oral and dental hygiene and cleaning agents according to the invention according to the invention that are preferred contain—based on their weight—0.01 to 2% by weight, preferably 0.05 to 1.5% by weight, more preferably 0.1 to 1% by weight, even more preferably 0.12 to 0.7% by weight and in particular 0.15 to 0.6% by weight cocoamidopropylbetaine.

According to the invention, the weight ratio of polishing agent(s) to surfactant(s) is ≦6, i.e., the polishing agents are used in a max. six-fold (weight) excess relative to the surfactants. The weight ratio is based on the weight ratio of all substances from the groups mentioned above to one another, wherein only the active substances are counted. For example, if an agent according to the invention includes 12% by weight polishing agent and 4% by weight of a 50% surfactant solution, then the weight ratio is 12/2=6. For example, if an agent according to the invention includes 12% by weight polishing agent and 4% by weight of a 50% surfactant solution+1% by weight pure sodium dodecyl sulfate, then the weight ratio is 12/(2+1)=4.

In the preferred agents according to the invention, the weight ratio is within an even narrower range. Especially preferred oral and dental hygiene and cleaning agents are characterized in that the weight ratio of polishing agent(s) to surfactant(s) is in the range of ≧5 to ≦15, preferably in the range of ≧7.5 to ≦12, more preferably in the range of ≧10 to ≦12 and in particular in the range of ≧10.25 to ≦11.9.

The agents according to the invention, in particular the toothpastes, may also includes substances to increase the mineralizing potential, for example, substances including calcium, e.g., calcium chloride, calcium acetate and dicalcium phosphate dihydrate. The concentration of the substance including calcium depends on the solubility of the substance and the interaction with the other substances present in the oral and dental hygiene agent.

In addition to the obligatory components mentioned above, the dental hygiene agents according to the invention may contain other essentially known excipients and additives. One additive that has been known as a toothpaste ingredient for a long time and is especially effective in the dental hygiene agents according to the invention is calcium glycerophosphate, which is the calcium salt of glycerol-1-phosphoric acid or glycerol-2-phosphoric acid or the glycerol-3-phosphoric acid that is an enantiomer to glycerol-1-phosphoric acid—or a mixture of these acids. This compound has a remineralizing effect in dental hygiene agents because it supplies both calcium ions and phosphate ions. In the dental hygiene agents according to the invention, calcium glycerophosphate is preferably used in amounts of 0.01-1% by weight. On the whole, the dental cleaning agents according to the invention may contain the usual excipients and additives in amounts up to 10% by weight.

The aromatic oils used here may include all the usual natural and synthetic flavorings used in oral and dental care agents. Natural flavorings may be used in the form of the natural essential oils isolated from herbs as well as the individual components isolated therefrom.

Suitable flavorings include, for example, peppermint oil, spearmint oil, eucalyptus oil, anise oil, fennel oil, caraway oil, menthyl acetate, cinnamaldehyde, anethol, vanillin, thymol as well as mixtures of these components.

Suitable sweeteners include sodium saccharin, sodium cyclamate, sucrose, lactose, maltose and fructose, for example.

Other conventional additives and excipients for toothpaste and mouthwash or mouth rinse solutions include:
surface-active substances, preferably anionic, zwitterionic, amphoteric, nonionic surfactants or a combination of several different surfactants,
solvents and solubilizers, for example, low monovalent or polyvalent alcohols or ethers, e.g., ethanol, 1,2-propylene glycol, diethylene glycol or butyl diglycol,
pigments such as titanium dioxide,
dyes,
buffer substances, e.g., primary, secondary or tertiary alkali phosphates or citric acid/sodium citrate,
other wound-healing or anti-inflammatory substances, e.g., allantoin, urea, azulene, chamomile active ingredients, acetylsalicylic acid derivatives or thiocyanates,
additional vitamins such as ascorbic acid, biotin, tocopherol or rutin,
mineral salts such as manganese, zinc or magnesium salts.

It has been found that the efficiency of the oral and dental hygiene and cleaning agents according to the invention can be further increased if the agents contain salivation-promoting substances. In particular the antibacterial effect and, with it, the anti-caries effect and the effects against gingivitis and/or periodontitis are strengthened in this way.

Salivation is understood to be the production and release of saliva, including non-physiologically elevated levels in the broader sense. Substances that stimulate salivation and increase the amount and/or release of saliva may originate from a wide variety of different classes of substances.

One example of a substance that is suitable according to the invention is pilocarpine which may be present in the oral and dental hygiene and cleaning agents according to the invention.

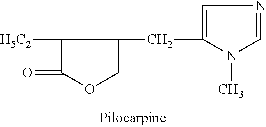

Additional salivation-promoting substances include in particular so-called sharp substances, i.e., sharp tasting substances and/or those that create a feeling of heat. Preferred oral and dental hygiene and cleaning agents according to the invention are characterized in that they contain at least one sharp tasting substance and/or a substance that imparts a feeling of heat as the salivation-promoting substance.

The products of this embodiment according to the invention contain a sharp-tasting substance and/or a substance that imparts a feeling of heat as the salivation-promoting ingredient. These substances impart to the user a sharp, tingling, mouth-watering or heat-producing effect, i.e., they give a sensory impression of heat or burning or a prickling, bubbling, tickling or fizzing and thereby promote salivation.

Products of this embodiment that are preferred according to the invention contain the sharp-tasting substances and/or substances that create a feeling of heat in amounts of 0.00001 to 5% by weight, preferably from 0.0005 to 2.5% by weight, more preferably from 0.001 to 1% by weight, especially preferably from 0.005 to 0.75% by weight and in particular from 0.01 to 0.5% by weight, each based on the weight of the total agent.

A number of substances may be used as a sharp-tasting substance or a substance that creates a feeling of heat. N-Alkyl-substituted amides of unsaturated carboxylic acids, for example, the following are preferred in particular:
2E,4E-decadienoic acid N-methylamide
2E,4E-decadienoic acid N-ethylamide
2E,4E-decadienoic acid N-n-propylamide
2E,4E-decadienoic acid N-isopropylamide
2E,4E-decadienoic acid N-n-butylamide
2E,4E-decadienoic acid N-(1-methylpropyl)amide
2E,4E-decadienoic acid N-isobutylamide
2E,4E-decadienoic acid N-tert-butylamide
2E,4Z-decadienoic acid N-methylamide
2E,4Z-decadienoic acid N-ethylamide
2E,4Z-decadienoic acid N-n-propylamide
2E,4Z-decadienoic acid N-isopropylamide
2E,4Z-decadienoic acid N-n-butylamide
2E,4Z-decadienoic acid N-(1-methylpropyl)amide
2E,4Z-decadienoic acid N-isobutylamide
2E,4Z-decadienoic acid N-tert-butylamide
2E,4E,8Z-decatrienoic acid N-methylamide
2E,4E,8Z-decatrienoic acid N-ethylamide
2E,4E,8Z-decatrienoic acid N-n-propylamide
2E,4E,8Z-decatrienoic acid N-isopropylamide
2E,4E,8Z-decatrienoic acid N-n-butylamide
2E,4E,8Z-decatrienoic acid N-(1-methylpropyl)amide
2E,4E,8Z-decatrienoic acid N-isobutylamide
2E,4E,8Z-decatrienoic acid N-tert-butylamide
2E,4Z,8Z-decatrienoic acid N-methylamide
2E,4Z,8Z-decatrienoic acid N-ethylamide
2E,4Z,8Z-decatrienoic acid N-n-propylamide
2E,4Z,8Z-decatrienoic acid N-isopropylamide
2E,4Z,8Z-decatrienoic acid N-n-butylamide
2E,4Z,8Z-decatrienoic acid N-(1-methylpropyl)amide
2E,4Z,8Z-decatrienoic acid N-isobutylamide
2E,4Z,8Z-decatrienoic acid N-tert-butylamide
2E,4E,8E-decatrienoic acid N-methylamide
2E,4E,8E-decatrienoic acid N-ethylamide
2E,4E,8E-decatrienoic acid N-n-propylamide
2E,4E,8E-decatrienoic acid N-isopropylamide
2E,4E,8E-decatrienoic acid N-n-butylamide
2E,4E,8E-decatrienoic acid N-(1-methylpropyl)amide
2E,4E,8E-decatrienoic acid N-isobutylamide
2E,4E,8E-decatrienoic acid N-tert-butylamide
2E,4Z,8E-decatrienoic acid N-methylamide
2E,4Z,8E-decatrienoic acid N-ethylamide
2E,4Z,8E-decatrienoic acid N-n-propylamide
2E,4Z,8E-decatrienoic acid N-isopropylamide
2E,4Z,8E-decatrienoic acid N-n-butylamide
2E,4Z,8E-decatrienoic acid N-(1-methylpropyl)amide
2E,4Z,8E-decatrienoic acid N-isobutylamide
2E,4Z,8E-decatrienoic acid N-tert-butylamide
2E,6Z,8E-decatrienoic acid N-methylamide
2E,6Z,8E-decatrienoic acid N-ethylamide
2E,6Z,8E-decatrienoic acid N-n-propylamide
2E,6Z,8E-decatrienoic acid N-isopropylamide
2E,6Z,8E-decatrienoic acid N-n-butylamide
2E,6Z,8E-decatrienoic acid N-(1-methylpropyl)amide
2E,6Z,8E-decatrienoic acid N-isobutylamide
2E,6Z,8E-decatrienoic acid N-tert-butylamide
2E,6E,8E-decatrienoic acid N-methylamide
2E,6E,8E-decatrienoic acid N-ethylamide
2E,6E,8E-decatrienoic acid N-n-propylamide
2E,6E,8E-decatrienoic acid N-isopropylamide
2E,6E,8E-decatrienoic acid N-n-butylamide
2E,6E,8E-decatrienoic acid N-(1-methylpropyl)amide
2E,6E,8E-decatrienoic acid N-isobutylamide
2E,6E,8E-decatrienoic acid N-tert-butylamide
2E,6Z,8Z-decatrienoic acid N-methylamide
2E,6Z,8Z-decatrienoic acid N-ethylamide
2E,6Z,8Z-decatrienoic acid N-n-propylamide
2E,6Z,8Z-decatrienoic acid N-isopropylamide
2E,6Z,8Z-decatrienoic acid N-n-butylamide
2E,6Z,8Z-decatrienoic acid N-(1-methylpropyl)amide
2E,6Z,8Z-decatrienoic acid N-isobutylamide
2E,6Z,8Z-decatrienoic acid N-tert-butylamide
2E,6E,8Z-decatrienoic acid N-methylamide
2E,6E,8Z-decatrienoic acid N-ethylamide
2E,6E,8Z-decatrienoic acid N-n-propylamide
2E,6E,8Z-decatrienoic acid N-isopropylamide
2E,6E,8Z-decatrienoic acid N-n-butylamide
2E,6E,8Z-decatrienoic acid N-(1-methylpropyl)amide
2E,6E,8Z-decatrienoic acid N-isobutylamide
2E,6E,8Z-decatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
02E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide
2E,7E,9E-undecatrienoic acid N-methylamide
2E,7E,9E-undecatrienoic acid N-ethylamide
2E,7E,9E-undecatrienoic acid N-n-propylamide
2E,7E,9E-undecatrienoic acid N-isopropylamide
2E,7E,9E-undecatrienoic acid N-n-butylamide
2E,7E,9E-undecatrienoic acid N-(1-methylpropyl)amide
2E,7E,9E-undecatrienoic acid N-isobutylamide
2E,7E,9E-undecatrienoic acid N-tert-butylamide
2E,7Z,9Z-undecatrienoic acid N-methylamide
2E,7Z,9Z-undecatrienoic acid N-ethylamide
2E,7Z,9Z-undecatrienoic acid N-n-propylamide
2E,7Z,9Z-undecatrienoic acid N-isopropylamide
2E,7Z,9Z-undecatrienoic acid N-n-butylamide
2E,7Z,9Z-undecatrienoic acid N-(1-methylpropyl)amide
2E,7Z,9Z-undecatrienoic acid N-isobutylamide
2E,7Z,9Z-undecatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
2E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide

Other substitution patterns on the nitrogen atom are of course also possible and preferred. For example, longer-chain n-alkyl radicals ( . . . -N-n-pentylamide, . . . -N-n-pentylamide, . . . -N-n-pentylamide, . . . -N-n-pentylamide, . . . -N-n-pentylamide, . . . -N-n-hexylamide, . . . -N-n-heptylamide, . . . -N-n-octylamide, . . . -N-n-nonylamide, . . . -N-n-decylamide, . . . -N-n-undecylamide, . . . -N-n-dodecylamide, . . . -N-n-tridecylamide, etc.) or disubstituted . . . -N,N-dialkylamides such as . . . -N,N-dimethylamide, . . . -N,N-diethylamide, . . . -N,N-di-n-propylamide, . . . -N,N-diisopropylamide, . . . -N,N-di-n-butylamide, . . . -N,N-di(1-methylpropyl)amide, . . . -N,N-diisobutylamide, . . . -N,N-di-tert-butylamide, . . . -N,N-methylethylamide, . . . -N,N-methyl-n-propylamide, . . . -N,N-methylisopropylamide, . . . -N,N-ethyl-n-propylamide, . . . -N,N-ethylisopropylamide, etc.

Of the compounds listed, a few of them are especially preferred within the scope of the present invention. These are listed below:

### 2E,6Z,8E-decatrienoic acid N-isobutylamide (N-isobutyl-2E,6Z,8E-decatrienamide, also known as spilanthol or affinin)

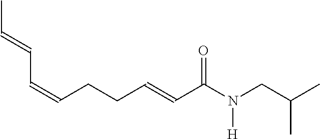 [SMILES: C/C=C/C=C\CC/C=C/C(=O)NCC(C)C]

### 2E,4E,8Z-decatrienoic acid N-isobutylamide (N-isobutyl-2E,4E,8Z-decatrienamide, also known as isoaffinin)

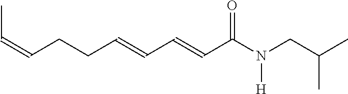 [SMILES: C/C=C\CC/C=C/C=C/C(=O)NCC(C)C]

### 2E,7Z,9E-undecatrienoic acid N-isobutylamide (N-isobutyl-2E,7Z,9E-undecatrienamide)

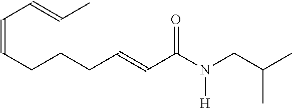 [SMILES: C/C=C/C=C\CCC/C=C/C(=O)NCC(C)C]

### 2E,4Z-decadienoic acid N-isobutylamide (cis-pellitorin)

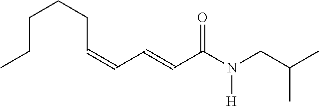 [SMILES: CCCCC/C=C\C=C\C(=O)NCC(C)C]

### 2E,4E-decadienoic acid N-isobutylamide (trans-pellitorin)

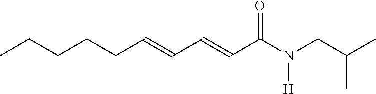 [SMILES: CCCCC/C=C/C=C/C(=O)NCC(C)C]

ferulic acid amides, for example,
ferulic acid N-vanillylamide:

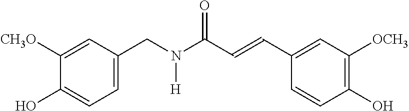

### N-[2-(4-hydroxy-3-methoxyphenyl)ethyl]-3-(4-hydroxy-3-methoxyphenyl)-(2E)-propenoic acid amide (trans-feruloyl methoxytyramine)

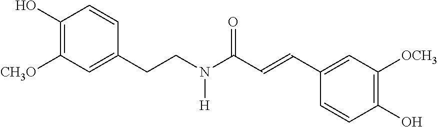

### N-[2-(4-hydroxy-3-methoxyphenyl)ethyl]-3-(4-hydroxy-3-methoxyphenyl)-(2Z)-propenoic acid amide (cis-feruloyl methoxytyramine)

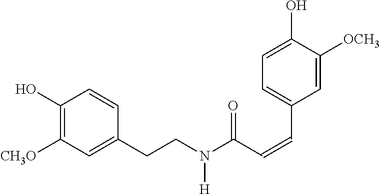

### N-[2-(4-hydroxy-3-methoxyphenyl)ethyl]-3-(4-hydroxy-3-methoxyphenyl)propanoic acid amide (dihydroferuloyl methoxytyramine)

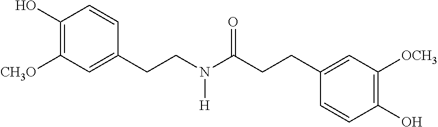

### N-[2-(3,4-dihydroxyphenyl)ethyl]-3-(4-hydroxy-3-methoxyphenyl)-(2E)-propenoic acid amide (trans-feruloyl dopamine)

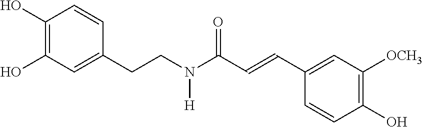 [SMILES: COc1cc(/C=C/C(=O)NCCc2ccc(O)c(O)c2)ccc1O]

### N-[2-(3,4-dihydroxyphenyl)ethyl]-3-(4-hydroxy-3-methoxyphenyl)-(2Z)-propenoic acid amide (cis-feruloyl dopamine)

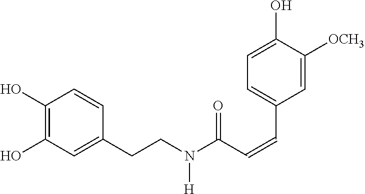 [SMILES: COc1cc(/C=C\C(=O)NCCc2ccc(O)c(O)c2)ccc1O]

### N-[2-(4-hydroxyphenyl)ethyl]-3-(3,4-dihydroxyphenyl)-(2E)-propenoic acid (trans-caffeoyltyramine)

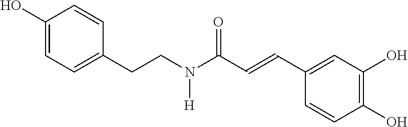 [SMILES: O=C(/C=C/c1ccc(O)c(O)c1)NCCc1ccc(O)cc1]

### N-[2-(4-hydroxyphenyl)ethyl]-3-(3,4-dihydroxyphenyl)-(2Z)-propenoic acid amide (cis-caffeoyltyramine)

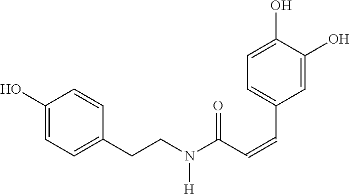 [SMILES: O=C(/C=C\c1ccc(O)c(O)c1)NCCc1ccc(O)cc1]

### N-[2-(3,4-dimethoxyphenyl)ethyl]-3-(3,4-dimethoxyphenyl)-(2E)-propenoic acid amide (trans-rubenamine)

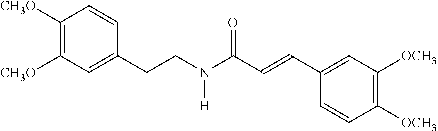

### N-[2-(3,4-dimethoxyphenyl)ethyl]-3-(3,4-dimethoxyphenyl)-(2Z)-propenoic acid amide (cis-rubenamine)

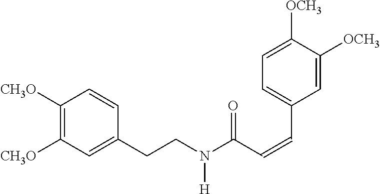

Additional sharp substances that can be used with particular preference within the scope of the present invention include, for example, extracts of natural plants. Sharp-tasting plant extracts may include all physiologically safe plant extracts that cause a sharp or warm sensory impression. Sharp-tasting plant extracts that are preferred include, for example, pepper extract (Piper ssp., in particular Piper nigrum), water pepper extract (Polygonum ssp., in particular Polygonum hydropiper), extracts of Allium ssp. (in particular onion and garlic extracts), radish extracts (Raphanus ssp.), horseradish extracts (Cochlearia armoracia), black mustard extracts (Brassica nigra), wild or yellow mustard (Sinapis ssp., in particular Sinapis arvensis and Sinapis alba), bertram root extract (Anacyclus ssp., in particular Anacyclus pyrethrum L.), echinacea extracts (Echinaceae ssp.), Szechuan pepper extracts (Zanthoxylum ssp., in particular Zanthoxylum piperitum), spilanthes extract (Spilanthes ssp., in particular Spilanthes acmella), chili extract (Capsicum ssp., in particular Capsicum frutescens), grains of paradise extract (Aframomum ssp., in particular Aframomum melegueta [Rose] K. Schum.), ginger extract (Zingiber ssp., in particular Zingiber officinale) and galanga extract (Kaempferia galanga or Alpinia galanga).

Gingerol obtained from ginger extract is an especially suitable substance:

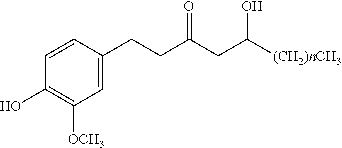

### N-Ethyl-p-menthane-3-carboxamide (N-ethyl-5-methyl-2-isopropylcyclohexanecarboxamide) can also be used

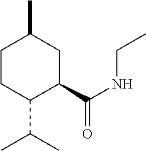

Other substances that have a sharp taste or create a feeling of heat may include, for example, capsaicin, dihydrocapsaicin, gingerol, paradol, shogaol, piperine, carboxylic acid N-vanillylamides, in particular nonanoic acid N-vanillylamide, 2-alkenoic acid amides, in particular 2-nonenoic acid N-isobutylamide, 2-nonenoic acid N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, nicotinaldehyde, methyl nicotinate, propyl nicotinate, 2-butoxyethylnicotinate, benzyl nicotinate, 1-acetoxychavicol, polygodial or isodrimeninol.

Preferred remineralizing products according to the invention are characterized in that they contain at least one sharp substance from the group of N-alkyl-substituted amides of unsaturated carboxylic acids, preferably
a. 2E,6Z,8E-decatrienoic acid N-isobutylamide (spilanthol) and/or
b. 2E,4E,8Z-decatrienoic acid N-isobutylamide and/or
c. 2E,7Z,9E-undecatrienoic acid N-isobutylamide and/or
d. 2E,4Z-decadienoic acid N-isobutylamide (cis-pellitorin) and/or
e. 2E,4E-decadienoic acid N-isobutylamide (trans-pellitorin) and/or
f. Ferulic acid N-vanillylamide and/or
g. N-[2-(4-hydroxy-3-methoxyphenyl)ethyl]-3-(4-hydroxy-3-methoxyphenyl)-(2E)-propenoic acid amide (trans-feruloyl methoxytyramine) and/or
h. N-[2-(4-hydroxy-3-methoxyphenyl)ethyl]-3-(4-hydroxy-3-methoxyphenyl)-(2Z)-propenoic acid amide (cis-feruloyl methoxytyramine) and/or
i. N-[2-(4-hydroxy-3-methoxyphenyl)ethyl]-3-(4-hydroxy-3-methoxyphenyl)propanoic acid amide (dihydroferuloyl methoxytyramine) and/or
j. N-[2-(3,4-dihydroxyphenyl)ethyl]-3-(4-hydroxy-3-methoxyphenyl)-(2E)-propenoic acid amide (trans-feruloyl dopamine) and/or
k. N-[2-(3,4-dihydroxyphenyl)ethyl]-3-(4-hydroxy-3-methoxyphenyl)-(2Z)-propenoic acid amide (cis-feruloyl dopamine) and/or
l. N-[2-(4-hydroxyphenyl)ethyl]-3-(3,4-dihydroxyphenyl)-(2E)-propenoic acid (trans-caffeoyltyramine) and/or
m. N-[2-(4-hydroxyphenyl)ethyl]-3-(3,4-dihydroxyphenyl)-(2Z)-propenoic acid amide (cis-caffeoyltyramine) and/or
n. N-[2-(3,4-dimethoxyphenyl)ethyl]-3-(3,4-dimethoxyphenyl)-(2E)-propenoic acid amide (trans-rubenamine) and/or
o. N-[2-(3,4-dimethoxyphenyl)ethyl]-3-(3,4-dimethoxyphenyl)-(2Z)-propenoic acid amide (cis-rubenamine).

In addition to or instead of the sharp substances mentioned above, other sharp-tasting substances or those that produce a feeling of heat may also be incorporated into the products according to the invention.

Within the scope of the present invention, alkyl-substituted dioxanes of the following formula have proven to be especially suitable:

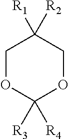

where R₁ and R₂ independently of one another are selected from —H, —CH₃, —CH₂CH₃ and R₃ and R₄ independently of one another are selected from —H, —CH₃, —CH₂CH₃, —CH₂CH₂CH₃, —CH(CH₃)₂.

Within the scope of the present invention, phenyl esters of the following formula have also proven to be especially suitable:

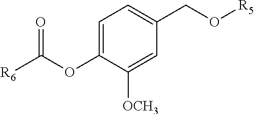

where R₅ stands for —CH₃ or a linear or branched alkyl or alkenyl radical with 2 to 8 carbon atoms and R₆ stands for —CH₃ or a linear or branched alkyl or alkenyl radical with 2 to 8 carbon atoms or an alkoxy group with 1 to 3 carbon atoms.

Within the scope of the present invention, carvon acetals of the following formula have also proven to be especially suitable:

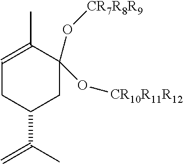

where R₇ to R₁₂ independently of one another are selected from —H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃ or R₉ and R₁₀ together form a chemical bond or denote a group —(CR13R14)ₓ, where x stands for the values 1 or 2 and R13 and R14 independently of one another are selected from
—H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃.

Agents according to the invention can be formulated as toothpastes or toothcreams. Another subject matter of the present invention is the use of agents according to the invention for cleaning teeth by means of electric toothbrushes.

Another subject matter of the present invention is a method for cleaning teeth, characterized in that an agent according to the invention is applied to the brush head of an electric toothbrush and the teeth are cleaned with the electric toothbrush.

Another subject matter is a method for cleaning teeth, which is characterized by the steps:
a) providing a toothbrush whose brush head can be set in motion electrically;
b) applying 0.5 to 5 g of an agent according to the invention to the brush head,
c) brushing the teeth for 30 to 300 seconds using the agent according to the invention with the use of the electrically operated brush head.

With respect to preferred embodiments of use according to the invention and the method according to the invention, what was said about the agents according to the invention is also true mutatis mutandis.

The compositions according to the invention can also be formulated as mouth rinse solutions or as mouthwash. Another subject matter of the present invention is a method for preventing and treating caries and/or combatting halitosis and/or for treatment of gingivitis or periodontitis in which a preparation according to the invention in the form of a mouth rinse solution is introduced into the oral cavity, where it is left for a period of at least 10 seconds, preferably at least 20 seconds and in particular at least 45 seconds.

Due to the use of the polylactic acid particles, the deposition of fluoride can be increased synergistically. Therefore another subject matter of the present invention is the use of polylactic acid particles to increase fluoride deposition.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oral and dental hygiene and cleaning agent, comprising, based on its weight,
a) 0.001 to 25 wt % polylactic acid particles, and
b) 1200 to 1600 ppm fluoride.

2. The oral and dental hygiene and cleaning agent according to claim 1, wherein the polylactic acid particles comprise 0.002-20% by weight, of the total weight of the agent.

3. The oral and dental hygiene and cleaning agent according to claim 1, wherein the polylactic acid particles comprise 0.003-17.5% by weight of the total weight of the agent.

4. The oral and dental hygiene and cleaning agent according to claim 1, wherein the polylactic acid particles comprise 0.005-12.5% by weight of the total weight of the agent.

5. The oral and dental hygiene and cleaning agent according to claim 1, wherein the polylactic acid particles have particle sizes of 1 to 1000 μm.

6. The oral and dental hygiene and cleaning agent according to claim 1 wherein the polylactic acid particles have particle sizes of 2 to 750 μm.

7. The oral and dental hygiene and cleaning agent according to claim 1, wherein the polylactic acid particles have particle sizes of 10 to 500 μm.

8. The oral and dental hygiene and cleaning agent according to claim 1, wherein the polylactic acid particles have average ECD values of 10 to 1000 μm.

9. The oral and dental hygiene and cleaning agent according to claim 1 wherein the polylactic acid particles have average ECD values of 50 to 500 μm

10. The oral and dental hygiene and cleaning agent according to claim 1, wherein the polylactic acid particles have average ECD values of 150 to 250 μm.

11. The oral and dental hygiene and cleaning agent according to claim 1, wherein the polylactic acid particles have an average solidity of 0.4 to 0.9.

12. The oral and dental hygiene and cleaning agent according to claim 1, wherein the polylactic acid particles have an average solidity of 0.5 to 0.8.

13. The oral and dental hygiene and cleaning agent according to claim 1, wherein the polylactic acid particles have an average solidity of 0.55 to 0.65.

14. The oral and dental hygiene and cleaning agent according to claim 1, wherein the the polylactic acid particles have an average circularity C of 0.1 to 0.6.

15. The oral and dental hygiene and cleaning agent according to claim 1, wherein the the polylactic acid particles have an average circularity C of 0.2 to 0.35.

16. The oral and dental hygiene and cleaning agent according to claim 1, wherein the fluoride is present in the agent in an amount of 1225 to 1575 ppm.

17. The oral and dental hygiene and cleaning agent according to claim 1, further comprising 5 to 25% by weight of a polishing agent selected from the group of silicic acids, aluminum hydroxide, aluminum oxide, dicalcium phosphate dihydrate (CaHPO₄.2H₂O) or mixtures of thereof.

18. The oral and dental hygiene and cleaning agent according to claim 1, further comprising 0.25 to 4% by weight, of anionic surfactant.

19. The oral and dental hygiene and cleaning agent according to claim 1, wherein the weight ratio of polishing agent(s) to surfactant(s) is ≧5 to ≦15.

20. A method for cleaning teeth, comprising the steps:
a) providing a toothbrush whose brush head can be induced electrically to movement;
b) applying 0.5 to 5 g of an agent according to claim 1 to the brush head,
c) tooth brushing for 30 to 300 seconds using the agent.
